(19) United States
(12) Patent Application Publication
(10) Pub. No.: US 2020/0104769 A1
(43) Pub. Date: Apr. 2, 2020

(54) ARTIFICIAL INTELLIGENCE PLANNING INSTANTIATION USING NATURAL LANGUAGE PROCESSING

(71) Applicant: INTERNATIONAL BUSINESS MACHINES CORPORATION, Armonk, NY (US)

(72) Inventors: Jonathan Bnayahu, Haifa (IL); Michael Katz, Elmsford, NY (US); Dany Moshkovich, Yokneam Ilit (IL); Muhammed Salamah, HAIFA (IL); Yan Tsitrin, HAIFA (IL)

(21) Appl. No.: 16/144,099

(22) Filed: Sep. 27, 2018

(51) Int. Cl. G06Q 10/06; G06F 17/30; G06F 17/27; G16H 10/60

(52) U.S. Cl. CPC G06Q 10/06311; G16H 10/60; G06F 17/27; G06F 17/30734

(57) ABSTRACT

A method for natural language instantiation of a planning problem includes receiving, by a computer, a natural language representation of a user's schedule, the user's schedule including a plurality of activities, associating a plurality of biomedical parameters of the user with execution of the plurality of activities to determine an effect of each activity on the plurality of biomedical parameters, based on the association of the plurality of biomedical parameters with the execution of the plurality of activities and a predefined evaluation criteria, calculating a quality score for the user's schedule, optimizing the user's schedule by performing a local search, based on a generic ontology, automatically constructing an ontology including information associated with the plurality of activities and the plurality of biomedical parameters of the user, and based on the constructed ontology, processing the natural language representation of the user's schedule.

### BACKGROUND

The present invention generally relates to the field of artificial intelligence, and more particularly to a method, system and computer program product for automated planning and scheduling of human activities based on natural language processing.

Automated planning and scheduling, sometimes denoted as AI planning, is a branch of artificial intelligence that involves the realization of strategies or action sequences. Automated planning can be summarized as: given an initial state, a desired goal, and a set of possible actions, finding an optimal sequence of actions which leads from the initial state to a state satisfying the goal. Automated planning is often a complex task, involving solutions that have to be discovered and optimized in multidimensional space. Solutions usually resort to iterative trial and error processes that can be implemented in artificial intelligence. Accordingly, developing applications and business solutions to perform automated planning often requires scientific expertise in first order logic-based languages, and deep understanding of the algorithms that are used to solve such models.

### SUMMARY

The following embodiments and aspects thereof are described and illustrated in conjunction with systems, tools and methods which are meant to be exemplary and illustrative, not limiting in scope.

According to an embodiment a system for developing an automated application for natural language instantiation of a planning problem includes at least one hardware processor; and a non-transitory computer-readable storage medium having stored thereon program instructions, the program instructions executable by the at least one hardware processor to: provide a first Application Programming Interface (API) for developing an automated scheduling application configured to generate a schedule for a user including a plurality of activities, where said application is configured to maximize a quality score of said schedule based, at least in part, on evaluating a plurality of biomedical parameters of the user; provide a second API for automatically constructing a ontology which represents information relating to a particular set of said activities and said biomedical parameters, where said construction is based, at least in part, upon (i) a generic ontology which represents information relating to human activities and biomedical parameters, and (ii) an instance of said application relating to said particular set of said activities and said biomedical parameters; and provide a third API for developing a natural-language module configured to perform a natural language processing (NLP) task based, at least in part, on said ontology.

According to another embodiment, a software development kit for developing an automated application for natural language instantiation of a planning problem includes program code embodied on a non-transitory computer-readable storage medium, the program code executable by at least one hardware processor to: provide a first Application Programming Interface (API) for developing an automated scheduling application configured to generate a schedule for a user including a plurality of activities, where said application is configured to maximize a quality score of said schedule based, at least in part, on evaluating a plurality of biomedical parameters of the user; provide a second API for automatically constructing an ontology which represents information relating to a particular set of said activities and said biomedical parameters, where said construction is based upon (i) a generic ontology which represents information relating to human activities and biomedical parameters, and (ii) an instance of said application relating to said particular set of said activities and said biomedical parameters; and provide a third API for developing a natural-language module configured to parse an input in a natural language and translate it into computer instructions based, at least in part, on said ontology.

According to yet another embodiment, a method for developing an automated application for natural language instantiation of a planning problem includes operating at least one hardware processor for: providing a first Application Programming Interface (API) for developing an automated scheduling application configured to generate a schedule for a user including a plurality of activities, where said application is configured to maximize a quality score of said schedule based, at least in part, on evaluating a plurality of biomedical parameters of the user; providing a second API for automatically constructing an ontology which represents information relating to a particular set of said activities and said biomedical parameters, where said construction is based upon (i) a generic ontology which represents information relating to human activities and biomedical parameters, and (ii) an instance of said application relating to said particular set of said activities and said biomedical parameters; and providing a third API for developing a natural-language module configured to parse an input in a natural language and translate it into computer instructions based, at least in part, on said ontology.

In some embodiments, said maximizing of said quality score includes applying a local search heuristic configured to generate a set of candidate schedules within a neighborhood of predefined constraints and permitted modifications, where said application is further configured to select one of the candidate schedules, based, at least in part, upon said quality score.

In some embodiments, said particular set of said activities and said biomedical parameters includes activities and biomedical parameters related to one or more chronic medical conditions selected from the group consisting of: cardiovascular disease, dementia, kidney disease, and diabetes.

In some embodiments, said biomedical parameters are selected from the group consisting of: heart rate, blood pressure, blood sugar level, number of calories consumed, number of carbohydrates consumed, number of calories burned, and type and quantity of medication taken by the user.

In some embodiments, said generic ontology is constructed based, at least in part, on one or more lexical semantic databases.

In some embodiments, said NLP task is selected from the group consisting of: word sense disambiguation, named entity detection, named entity resolution, named entity inference, and word sense inference.

In some embodiments, said third API is further configured for developing a user-interface for said application, based, at least in part, on said NLP task.

In addition to the exemplary aspects and embodiments described above, further aspects and embodiments will become apparent by reference to the figures and by study of the following detailed description.

### BRIEF DESCRIPTION OF THE DRAWINGS

The following detailed description, given by way of example and not intended to limit the invention solely thereto, will best be appreciated in conjunction with the accompanying drawings, in which:

FIG. 1A is a block diagram of the functional elements of a method for natural language instantiation of planning problems for human activities affecting biomedical parameters of a user, according to an embodiment of the present disclosure;

FIG. 1B is a block diagram of the functional elements of a generic planning model, according to an embodiment of the present disclosure;

FIGS. 1C and 1D are snapshots of a user interface of a generic planning model, according to an embodiment of the present disclosure;

FIG. 2 is a schematic illustration of the process for creating a business-specific ontology, according to an embodiment of the present disclosure;

FIG. 3 is a schematic illustration of the process for translating a natural language phrase into computer instructions, according to an embodiment of the present disclosure;

FIG. 4 is a flowchart depicting the steps of the method for natural language instantiation of planning problems for human activities affecting biomedical parameters of the user, according to an embodiment of the present disclosure;

FIG. 5 is a block diagram of internal and external components of a generic computer system, according to an embodiment of the present disclosure;

FIG. 6 is a block diagram of an illustrative cloud computing environment, according to an embodiment of the present disclosure; and

FIG. 7 is a block diagram of functional layers of the illustrative cloud computing environment of FIG. 6, according to an embodiment of the present disclosure.

The drawings are not necessarily to scale. The drawings are merely schematic representations, not intended to portray specific parameters of the invention. The drawings are intended to depict only typical embodiments of the invention. In the drawings, like numbering represents like elements.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. In the description, details of well-known features and techniques may be omitted to avoid unnecessarily obscuring the presented embodiments.

Automated planning and scheduling, sometimes denoted as AI planning, is a branch of artificial intelligence that concerns the realization of strategies or action sequences. There are software platforms that allow software developers to rapidly create AI activity planning applications and solutions, for example, in the healthcare domain, which consider the effect of the planned activities on a user's biomedical parameters. However, these applications often rely on user's input for initiating the planning task, and therefore include specific user interfaces. The users of the applications are then faced with the task of learning to operate the different user interfaces, in order to make use of the applications. Accordingly, providing users with a simplified way to input data into activity planning applications may be of significant value.

In recent years, the field of software engineering has placed increasing emphasis on software reusability as a key to reducing the time and cost of application system construction and maintenance. For example, software frameworks have been developed which allow application developers to rapidly create activity planning applications in various domains from reusable building blocks. Such applications may be aimed at planning an schedule of activities associated with a user considering the effect of the activities on certain user's biomedical parameters, with a view to improving overall health and well-being. However, these applications may rely on user-input data, which is typically entered using traditional user interface methods that require manual typing and/or menu options selection.

The use of natural language processing (NLP) interfaces may have several advantages over these traditional user interface methods. For example, common interfaces require physical interaction with a device, which is difficult in eyes-busy/hands-busy situations. In contrast, an NLP interface does not require manual input and/or eye contact with the device, and can be used while performing other tasks, such as walking or driving. Using NLP can also reduce task-completion time. Moreover, various studies have shown that NLP is typically better at expressing certain questions/commands, such as those requiring negation, quantification, and temporal information.

NLP in a particular domain typically requires a semantic organization of concepts in that domain, so that it may be more readily expressed linguistically. One way to provide for such knowledge organization is by creating an ontology for the domain. In the field of computer science, and specifically, artificial intelligence, an ontology is a structured system of concepts covering the processes, objects, and attributes of a domain, as well as all their pertinent complex relationships. The various concepts (or classes) are structured in a hierarchy. Each concept in turn typically has various properties (also called attributes, slots, or roles), which describe the meaning and characteristics of the concept. Properties can then be used to describe the relationships between concepts in the ontology. For example, in an ontology of activities, one class may be “food consumption.” Subclasses of “food consumption” may include “meals,” “snacks,” etc. Properties, or ‘slots,’ of the class “food consumption” and its subclasses may include meal type, meal duration, quantity, time of day, etc. Accordingly, in practical terms, developing an ontology includes defining classes in the ontology, arranging the classes in a taxonomic (subclass—superclass) hierarchy, defining slots, describing allowed values for these slots, and, finally, filling in the values for specific instances. By combining all these elements, there can be created a knowledge base in that particular domain. The knowledge base, in turn, may be used to give meaning to natural language entries in the context of that domain, by providing a common understanding of concepts in the domain. As an example, some words may have different meanings in different domains; the word “mouse,” for instance, means one thing in an ontology of animals, and another in a computer hardware ontology. Ontologies thus enable ascribing a meaning to a word within a particular context, and can help bridge the representational gap between information input by a user via unstructured natural language and the structured information needs of a planning application.

As such, integrating an NLP capability into a customized case-specific planning application will require a suitable ontology in the specific subdomain. Although a more ‘generic’ or broader ontology can be used, different scheduling environments invariably present different challenges (e.g., different dominating constraints, different objectives, different domain structure, different sources of uncertainty, etc.). Accordingly, to enhance application performance, a customized solution may be required. However, the manual construction of a case-specific ontology for each case-specific application is an expensive and time-consuming proposition.

Embodiments of the present disclosure generally relate to the field of artificial intelligence, and more particularly to a method, system and computer program product for automated planning and scheduling of human activities based on natural language processing. Specifically, the following described exemplary embodiments provide a method system and computer program product for natural language instantiation of planning problems for human activities affecting biomedical parameters of a user. Therefore, the present embodiments have the capacity to improve the technical field of artificial intelligence by, at a minimum, combining artificial intelligence concepts in activity planning solutions, ontology acquisition, and natural language processing, to provide a toolkit that enhances both_developer productivity and end-user experience.

More specifically, embodiments of the present disclosure enable application users to enter information using natural language phrasing in order to make the data input task less onerous and time-consuming and improve overall user experience. Accordingly, developers of planning applications (e.g., in the healthcare domain) are capable of incorporating NLP capabilities without the need for specialized knowledge in artificial intelligence.

As such, a generic planning model including a computerized toolkit for creating applications directed at solving planning problems in the healthcare domain (for example, activity schedule optimization for improving bio-medical parameters) is provided. The generic planning model may be used for creating customized applications for case-specific planning problems (e.g., an activity schedule planning application specifically for diabetics).

Consequently, multiple customized ontologies can be automatically configured_for case-specific activity planning problems within the wider healthcare domain. The case-specific planning model can be used for deriving a case-specific ontology, by intersecting it with a provided ‘generic’ core ontology for planning problems in the healthcare domain. Once derived, the case-specific ontology may be used to facilitate the effective integration of an NLP interface in the case-specific planning application. The NLP interface may then be used for problem instantiation using standard human language input phrases, as will be described in detail below.

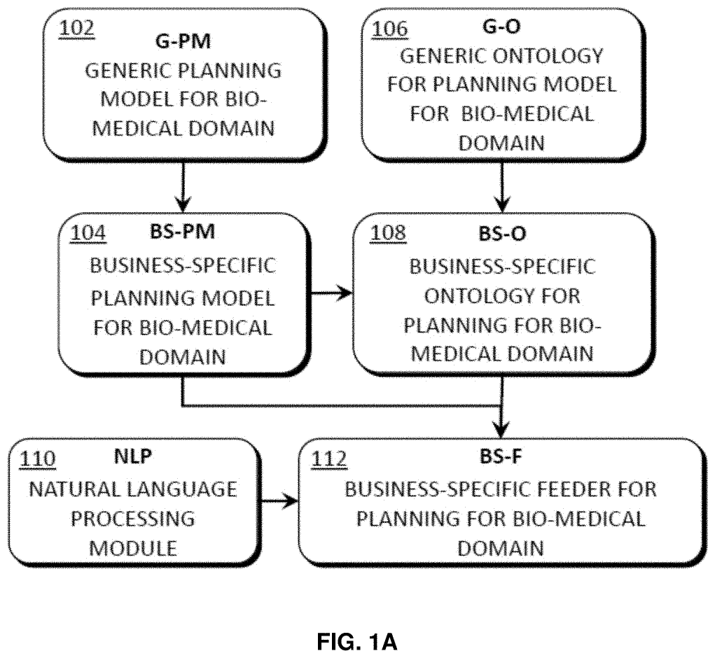

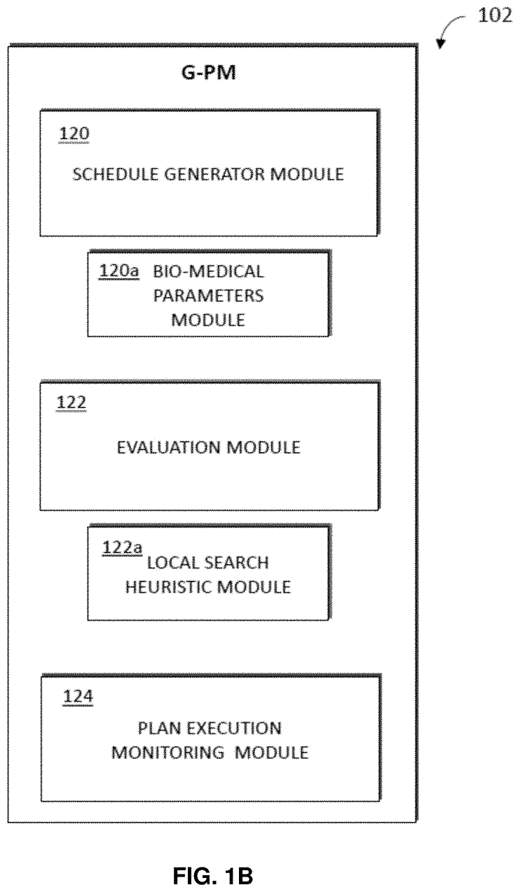

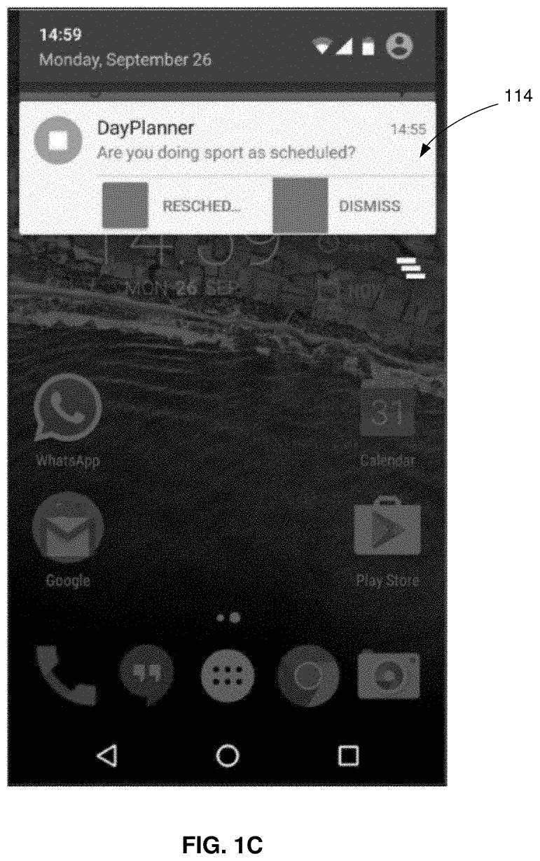

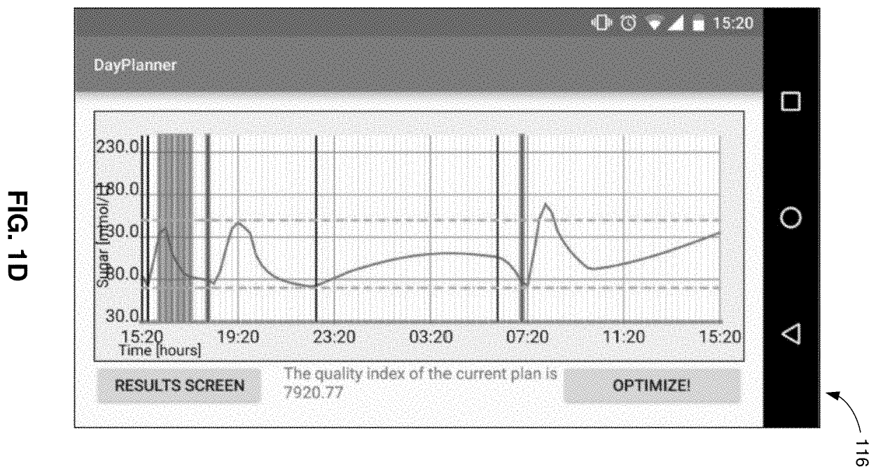

Referring now to FIG. 1A, a block diagram of functional elements is shown, according to an embodiment of the present disclosure. In this embodiment, a generic planning model module (G-PM) 102 including a set of computerized tools for the creation of applications for solving planning problems may be implemented in a computer language such as Java®. The G-PM module 102 may include several modules, libraries, and/or Application Programming Interfaces (API).

In some embodiments, the G-PM 102 may include an application having one or more modules which allow the creation of an automated and/or user-defined schedule of activities relevant to a healthcare-specific planning problem, as shown in FIG. 1B.

Referring now to FIG. 1B, the G-PM 102 may include, for example, a scheduling generator module 120 which allows the creation of an initial daily schedule for a diabetic, including activities such as meals, meetings, workout sessions, medical treatments, rest times, and the like. Each of these activities may be defined by a plurality of parameters and constraints, such as availability slot, activity time, duration, an importance attribute associated with the activity (e.g., optional, recommended, mandatory), and a numerical or quantitative value associated with the activity (e.g., whether a meal should be full or light, or whether a workout should be mild or intense). Some constraints may define, for example, relational attributes between two or more activities, such as dependency (e.g., an insulin injection activity can only be performed from 30 min to 10 min before a meal), and temporal ordering (before, during, after).

In some embodiments, the schedule generator module 120 may include a biomedical parameters module 120a, which may allow defining and associating biomedical parameters of a user with the execution by the user of a schedule of activities, thereby encoding the effect, over time, of these activities on the user's biomedical parameters and other well-being criteria. For example, the biomedical parameters module 120a may incorporate and take account of the effects, over time, of each activity in the schedule created by the G-PM 102 on a predefined set of biomedical measurements of a user, such as heart rate, blood pressure, blood sugar levels, and the like. Additional biomedical parameters (such as calories burned) may be defined in the biomedical parameters module 120a and incorporated through activity-specific evaluators. For example, the amount of calories burned can be derived from the type and duration of a physical activity, based on known human medical constants.

In some embodiments, the G-PM 102 may include an evaluation criteria module or simply evaluation module 122, configured for evaluating and optimizing an initial schedule generated by the schedule generator module 120 based on a weighted aggregation of evaluation criteria. Such (predefined) criteria may include a predefined set of criteria, such as time spent outside a predefined safe range for a given biomedical measurement, drug dosages consumed, deviation from original schedule, deviation from medical guidelines, etc. The evaluation module 122 may further include a local search heuristic module 122a, configured for modifying and optimizing an initial schedule generated by G-PM 102 within a defined set of allowed modifications, where an optimized plan is one which satisfies the evaluation criteria defined in the evaluation module 122. In some embodiments, the local search heuristic module 122a may include a heuristic search-based planning system, such as a local search algorithm. In many optimization problems, such as schedule planning, it is less important how the goal is reached, so long as it satisfies certain evaluation criteria. In such cases, a local search strategy can be used to find optimized solutions to the planning problem. Local search is an iterative algorithm that begins with an initial state, which can be user-determined or randomly chosen. A “neighborhood” of solutions is defined and built for the initial solution. The local search then picks a subsequent solution from the universe of neighboring solutions, and compares it to the initial solution based on one or more criteria. If the subsequent solution better satisfies the evaluation criteria compared to the initial solution, it is deemed to be the ‘current solution’ within the system, and another local search is performed, until the criteria is maximized, or a time or iterations limit has been reached. Accordingly, in some embodiments, the local search heuristic module 122a may employ a local search heuristic which searches for an optimum solution, where neighboring solutions are determined based on the allowed modifications space. The allowed modifications space can be determined using predefined default modifications and/or user-defined modifications. For example, in some embodiments, the local search heuristic module 122a may be configured for adjusting the modification space to a specific problem at hand, e.g., changing the start time of the event by a predefined time offset, increasing or reducing the amount of carbohydrates, adding or removing a certain activity, etc. Such allowed modifications to a schedule plan may include modifying a single activity or a collection of activities, using either predefined default modifications or user-defined modifications. In some embodiments, the program enforces a limit on the time of the search process, to facilitate its online usage.

In some embodiments, the local search heuristic module 122a may further be configured to employ restarts. Local search restarts may be employed in cases where no improving configurations are present in the neighborhood of solutions, and the local search may become stuck at a locally optimal or maximal point. A local-optima problem can be cured by using restarts, i.e., repeated local searches with different initial conditions. In some embodiments, the local search heuristic module 122a may be configured to employ random and heuristic-based restarts, such as restarts from historical schedules, whereby a new initial schedule is selected from previously-considered schedules in the user's history. Another potential restart may be based on a user-defined schedule, e.g., a schedule manually defined by a user, possibly by manually rescheduling, adding, and/or removing activities. A restart may also be based on a randomly-selected schedule, e.g., randomly-selected times for activities within the predefined time windows. Finally, a restart may be based on a schedule obtained by abstracting problem aspects, where the program abstracts away some restrictions that make a schedule valid, such as predefined time period between specified activities, etc.

Once all restarts have been exhausted the iterative process of module 122a ceases, module 122a may be configured for selecting an optimized plan as the final plan to be presented to the user.

In some embodiments, G-PM 102 may further include a plan execution monitoring module 124. After generating and optimizing the initial plan or schedule, the plan execution monitoring module 124 may be configured for monitoring, on an ongoing basis, the execution of the plan by a user. For example, as shown in FIG. 1C, the plan execution monitoring module 124 may include a user interface 114 through which the plan execution monitoring module 124 may prompt a user to report on the progress of one or more activities included in the plan. In some variations, the plan execution monitoring module 124 may be configured to allow the user to manually modify a parameter of the plan, e.g., reschedule a planned activity. In other variations, the plan execution monitoring module 124 may also receive input in real time regarding one or more biomedical parameters of the user, including, but not limited to, blood sugar levels, heart rate, blood pressure, and/or calories burned. User's biomedical input may be, e.g., reported by the user or otherwise received directly from one or more device communicatively connected to the G-PM 102. Based on the ongoing monitoring of the execution of the plan and user and/or other inputs, the plan execution monitoring module 124 may dynamically modify any then-current plan in real time, using the same iterative optimization process described above. FIG. 1D shows an exemplary results screen 116 after the dynamic optimization, based, for example, on user input or user-initiated modifications.

With continued reference to FIG. 1A, on the basis of the application tools provided by the G-PM 102, in some embodiments, a business-specific planning model (BS-PM) 104 may be developed, which is aimed at solving a specific planning problem in the healthcare domain. Some examples are optimizing schedules of activities for sufferers of chronic conditions, such as cardiovascular disease, dementia, kidney disease, or diabetes. For the purpose of this discussion, the present invention shall be described with respect to the case-specific problem of schedule planning for diabetics.

A case-specific planning application for diabetics may include a specific set of concepts which is relevant to that particular category. For example, activities that affect the well-being of diabetics may include food consumption, administration of insulin doses, and physical exercise. For each of these categories, there may be defined specific subcategories with associated attributes, values, and constraints. For example, a “food consumption” activity may be defined on the basis of a meal type (breakfast, lunch, dinner, and/or snack), time slot, duration, quantity (light or full meal), and/or expected caloric intake. The scheduling of these activities may then be subject to additional constraints, such as user availability; minimum-maximum activity duration ranges; minimum-maximum quantity ranges; technical details (e.g., quantity and type and of insulin injection—basal or bolus); a temporal precedence between two or more activities (before, after, or at the same time); a relationship between two or more activities (e.g., an insulin injection dose must be taken from 30 min to 10 min before a meal); and an importance attribute associated with an activity (e.g., optional, recommended, mandatory, etc.). As noted, the BS-PM 104 may further allow including definitions of biomedical parameters which are relevant to a user who is, for example, a diabetic. Such biomedical parameters may be input by a user and/or measured by a suitable device, and analyzed over time such that the application can evaluate the expected effect of a particular activity thereon. In some embodiments, such biomedical parameters may include heart rate, blood pressure, blood sugar level, number of calories consumed, number of carbohydrates consumed, and number of calories burned.

In some embodiments, a base core ontology (G-O) 106 for activity planning models that affect biomedical parameters may be developed or provided. As will be described below, in some embodiments, the G-O 106 is then used as the basis for the automated construction of a case-specific ontology to enable the usage of NLP in specific sub-categories of the healthcare domain.

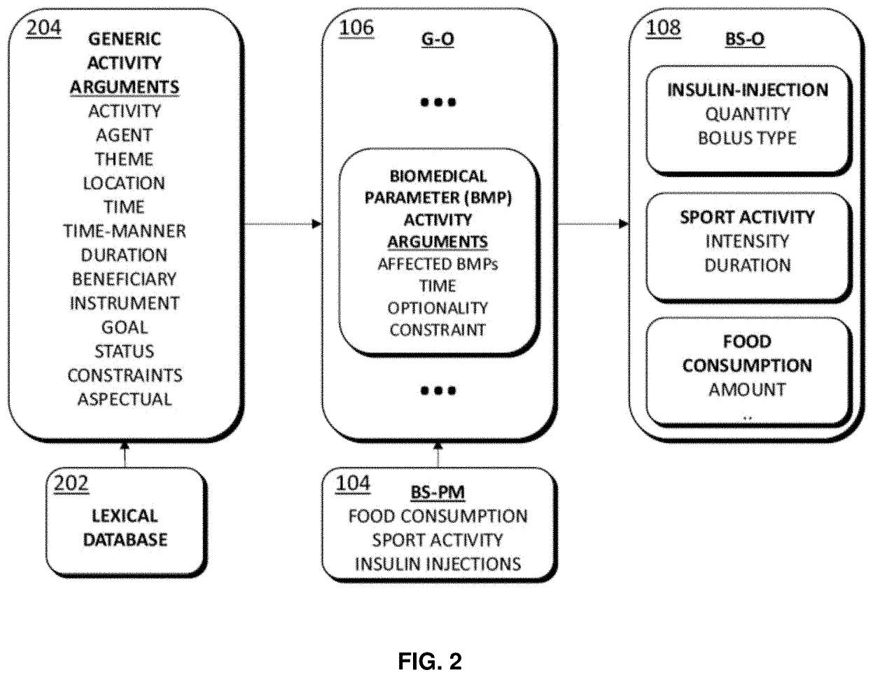

Referring now to FIG. 1A and FIG. 2, in some embodiments, the G-O 106 is constructed manually as an ontology for a generic activity planning problem, whereby the activities affect biomedical parameters. As shown in FIG. 2, the G-O 106 may begin with a base lexical semantic database 202, such as WordNet. WordNet is a lexical database of English, where nouns, verbs, adjectives and adverbs are grouped into sets of cognitive synonyms (synsets), each expressing a distinct concept. Synsets are interlinked by means of conceptual-semantic and lexical relations. In some embodiments, additional or other base lexical databases may be used.

The base lexical database may then be reduced to a list of generic activity arguments 204. The list of generic activity arguments 204 may then be modified, for example, by a process of identifying relevant concepts, resolving conflicts among concepts, removing irrelevant concepts, and establishing a taxonomy among concepts. At the conclusion of this process, G-O 106 includes a generic knowledge base for activities in the healthcare domain, which includes relevant arguments within that scope.

In some embodiments, a module for automated construction of a case- or business-specific ontology (BS-0) 108, based upon (i) a case-specific planning instance developed using BS-PM 104, and (ii) the core ontology G-O 106 may be provided, as shown in FIGS. 1A and 2. For example, by intersecting G-O 106 and concepts derived from BS-PM 104, activity arguments that are relevant to BS-PM 104 are acquired from G-O 106 into BS-O 108. In some embodiments, the ontology derivation and acquisition processes employed in the context of creating G-O 106 and/or BS-O 108 are based on known ontology acquisition methods.

With continued reference to FIG. 1A, once BS-O 108 is generated as described above, it can be used in conjunction with an NLP processing module 110. In some embodiments, the NLP processing module 110 is based on known NLP interface technologies, such as IBM® Watson® Conversation service.

A user of the application may then use an NLP interface created using the NLP processing module 110 to input natural language phrases such as:
[Tomorrow] I would like to have a [light] [breakfast] at [9 am].
I would like to have a [full] [lunch] sometime [around noon] and a [full dinner] sometime [between 6 pm and 7 pm].
I want to go to [gym] for an [intense] [workout] for [an hour], [if possible], [before dinner]. I want to have a [snack] [before gym].
I usually make my [basal insulin injection] at [8 pm].
Also, I have a [business meeting] between [1 pm and 4 pm].

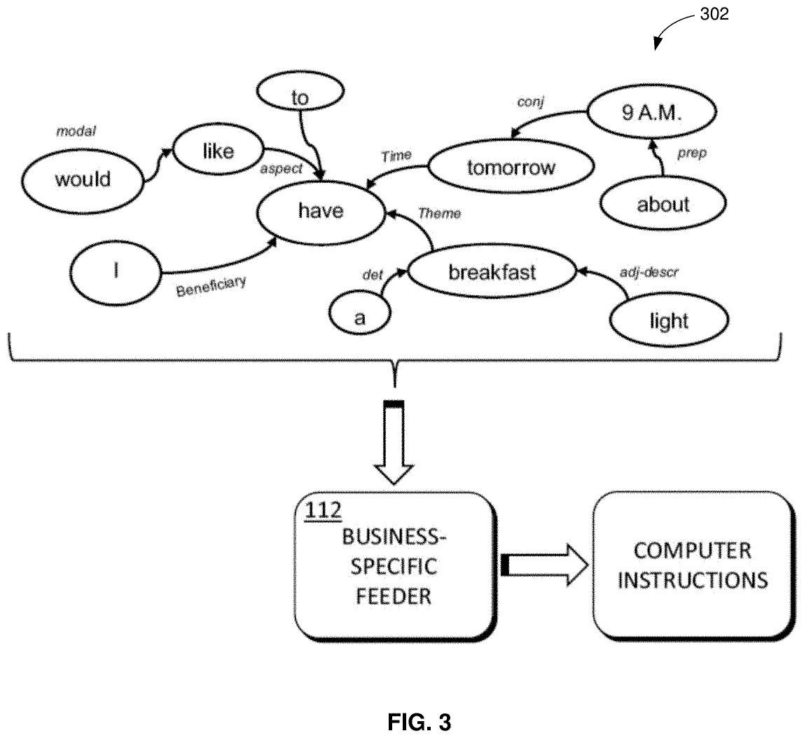

In each of these phrases, there are bracketed activity-related arguments that need to be construed in the context of the case-specific ontology BS-O 108 (FIG. 1A), and then translated into appropriate computer instructions. A Business-specific feeder module (BS-F) 112 then ‘translates’ natural language utterances 302, as schematically illustrated in FIG. 3. For example, the various elements of an utterance “[Tomorrow] I would like to have a [light] [breakfast] at [9 am]” are parsed such that individual elements and their relationships may be identified using BS-O 108. Accordingly, for example, the word “breakfast” is identified as a theme, with the word “light” as an adjective which modifies it. Exemplary computer instructions derived by BS-F 112 from the referenced utterance may then be as follows:
“activities”:[
{“activityId”: “breakfast”,
“activityType”: “meal”,
“startAfter”: “9 am”,
“startBefore”: “9 am”,
“mealType”: “light”}.

Similarly, the other phrases referenced above may be rendered in computer instructions by BS-F 112 as follows:
I would like to have a [full] [lunch] sometime [around noon] and a [full dinner] sometime [between 6 pm and 7 pm]:
{“activityId”: “lunch”,
“activityType”: “meal”,
“startAfter”: “11 am”,
“startBefore”: “1 pm”,
“mealType”: “full”},
{“activityId”: “dinner”,
“activityType”: “meal”,
“startAfter”: “6 pm”,
“startBefore”: “7 pm”,
“ mealType”: “full”},
I want to go to [gym] for an [intense] [workout] for [an hour], [if possible], [before dinner]. I want to have a [snack] [before gym]:
{“activityId”: “gym”,
“activityType”: “sport”,
“endBeforeActivity”: “dinner”,
“intensity”: “intense”,
“durationInMinutes”: “60”,
“optional”: “true”},
I usually make my [basal insulin injection] at [8 pm]:
{“activityId”: “basal insulin”,
“activityType”: “insulin”,
“startAfter”: “7 pm”,
“startBefore”: “9 pm”,
“insulinType”: “basal”},
Also, I have a [business meeting] between [1 pm and 4 pm]:
{“activityId”: “business meeting”,
“activityType”: “blocking”,
“startAfter”: “1 pm”,
“startBefore”: “1 pm”,
“durationInMinutes”: “180”}

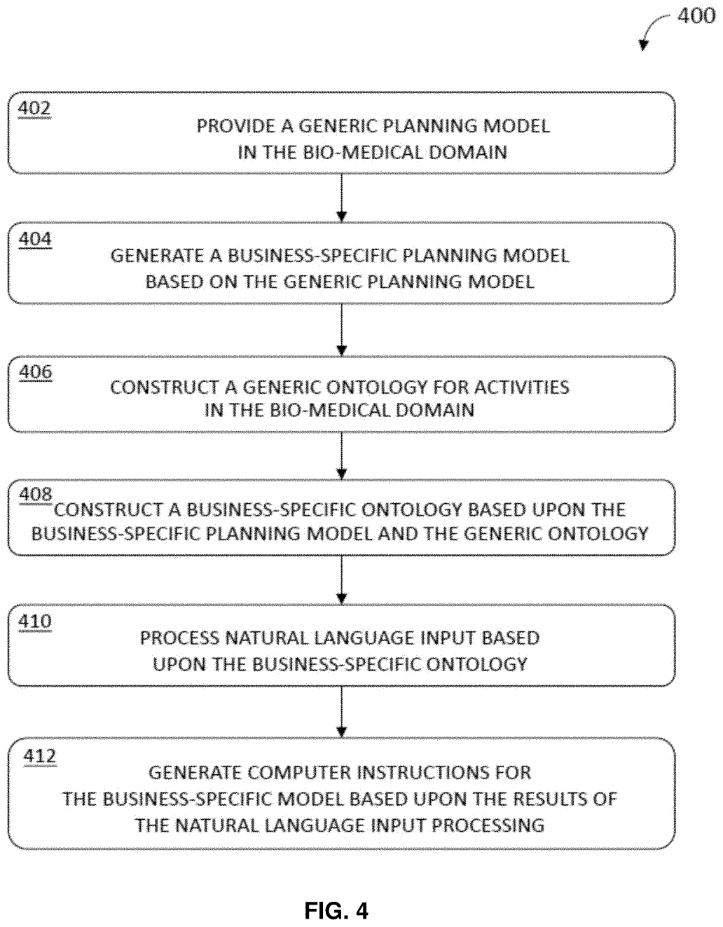

Referring now to FIG. 4, a flowchart 400 of the operational steps carried out by an application to create a schedule for a user within the healthcare domain using NLP capabilities is shown, according to an embodiment of the present disclosure. In some embodiments, the program is implemented as a mobile application running on a mobile computing device (for example, a smartphone, a wearable device, a tablet computer, a laptop computer, etc.). In other embodiments, the application may be running on a cloud infrastructure and is accessible by a user from various user devices through an interface, such as a web browser or a local software client.

At 402, a generic planning model in the biomedical domain is provided. At 404, the generic planning model is used for generating a business-specific planning model for a specific planning problem (e.g., planning a schedule of activities for a user with diabetes)

At 406, a generic ontology for activities in the biomedical domain is constructed. At 408, a business-specific ontology is constructed, based upon the business-specific planning model generated at 404 and the generic ontology constructed at 406.

At 410, a natural language input from a user is processed, based upon the business-specific ontology constructed at 408. The natural language input including a natural language representation of a user's schedule. At 412, the natural language input is translated into computer instructions for the business-specific model, based on the results of the natural language input processing, to instantiate an activity planning problem.

The previously described embodiments provide a method, system, and computer program product for automated planning and scheduling based on natural language processing. Embodiments of the present disclosure, support automatic and semi-automatic NLP-based instantiation of activity planning problems affecting biomedical parameters by targeting problems specified in a formal planning language, and describing a business specific planning model by extending the generic planning model. As such, embodiments of the present disclosure provide a generic ontology for a generic planning model, a method for semi-automatic generation of a business specific ontology from a business specific planning model based on the generic ontology, and a method for automated generation of a component for feeding the output of a standard NLP tool based on the provided business specific ontology into the business specific planning model.

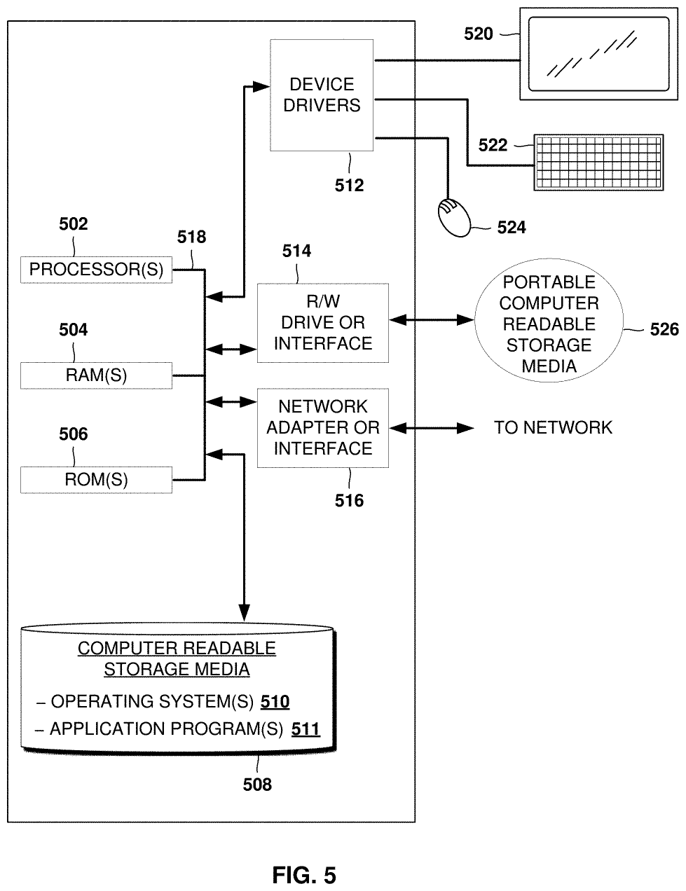

Referring now to FIG. 5, a block diagram 500 of internal and external components of a generic computer system in which embodiments of the present disclosure can be implemented is shown, according to an embodiment of the present disclosure. It should be appreciated that FIG. 5 provides only an illustration of one implementation and does not imply any limitations regarding the environments in which different embodiments may be implemented. Many modifications to the depicted environment may be made.

The generic computer system of FIG. 5 may include one or more processors 502, one or more computer-readable RAMs 504, one or more computer-readable ROMs 506, one or more computer readable storage media 508, device drivers 512, read/write drive or interface 514, network adapter or interface 516, all interconnected over a communications fabric 518. Communications fabric 518 may be implemented with any architecture designed for passing data and/or control information between processors (such as microprocessors, communications and network processors, etc.), system memory, peripheral devices, and any other hardware components within a system.

One or more operating systems 510, and one or more application programs 511, for example, the application to create a schedule for a user within the healthcare domain using NLP described in FIG. 4, are stored on one or more of the computer readable storage media 508 for execution by one or more of the processors 502 via one or more of the respective RAMs 404 (which typically include cache memory). In the illustrated embodiment, each of the computer readable storage media 508 may be a magnetic disk storage device of an internal hard drive, CD-ROM, DVD, memory stick, magnetic tape, magnetic disk, optical disk, a semiconductor storage device such as RAM, ROM, EPROM, flash memory or any other computer-readable tangible storage device that can store a computer program and digital information.

The generic computer system may also include a R/W drive or interface 514 to read from and write to one or more portable computer readable storage media 526. Application programs 511 may be stored on one or more of the portable computer readable storage media 526, read via the respective R/W drive or interface 514 and loaded into the respective computer readable storage media 508.

The generic computer system may also include a network adapter or interface 516, such as a TCP/IP adapter card or wireless communication adapter (such as a 4G wireless communication adapter using OFDMA technology) for connection to a network 528. Application programs 511, such as the application to create a schedule for a user within the healthcare domain using NLP described in FIG. 4, may be downloaded to the computing device from an external computer or external storage device via a network (for example, the Internet, a local area network or other wide area network or wireless network) and network adapter or interface 516. From the network adapter or interface 516, the programs may be loaded onto computer readable storage media 508. The network may comprise copper wires, optical fibers, wireless transmission, routers, firewalls, switches, gateway computers and/or edge servers.

The generic computer system may also include a display screen 520, a keyboard or keypad 522, and a computer mouse or touchpad 524. Device drivers 512 interface to display screen 520 for imaging, to keyboard or keypad 522, to computer mouse or touchpad 524, and/or to display screen 520 for pressure sensing of alphanumeric character entry and user selections. The device drivers 512, R/W drive or interface 514 and network adapter or interface 516 may comprise hardware and software (stored on computer readable storage media 508 and/or ROM 506).

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

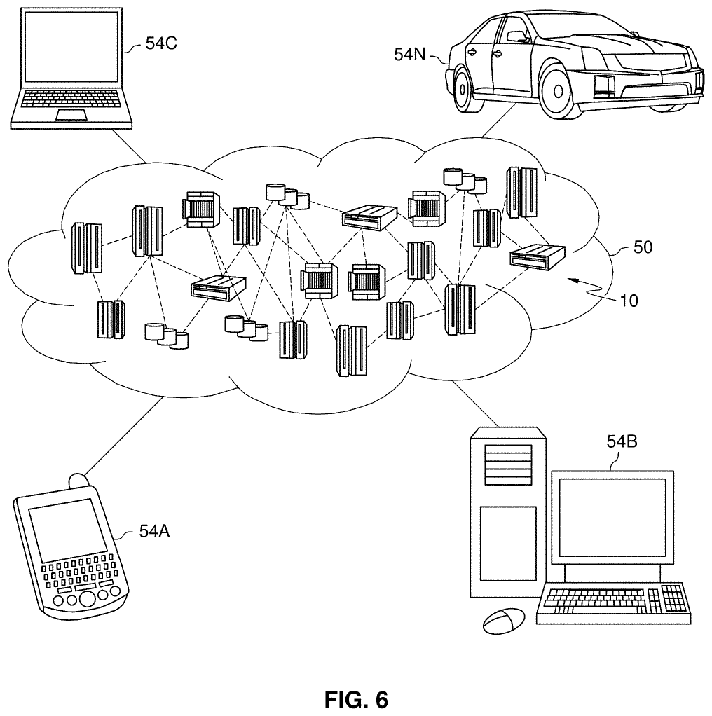

Referring now to FIG. 6, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 6 are intended to be illustrative only and that computing nodes 10 and cloud computing environment 50 can communicate with any type of computerized device over any type of network and/or network addressable connection (e.g., using a web browser).

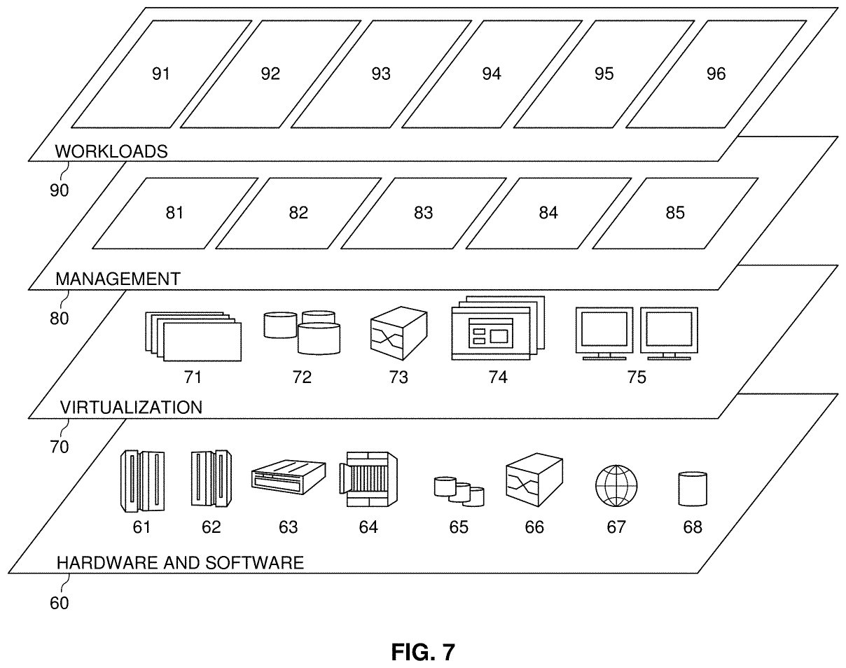

Referring now to FIG. 7, a set of functional abstraction layers provided by cloud computing environment 50 (FIG. 6) is shown. It should be understood in advance that the components, layers, and functions shown in FIG. 7 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and NLP-based activity planning 96.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While steps of the disclosed method and components of the disclosed systems and environments have been sequentially or serially identified using numbers and letters, such numbering or lettering is not an indication that such steps must be performed in the order recited, and is merely provided to facilitate clear referencing of the method's steps. Furthermore, steps of the method may be performed in parallel to perform their described functionality.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for natural language instantiation of a planning problem, the method comprising:
receiving, by a computer, a natural language representation of a user's schedule, the user's schedule comprising a plurality of activities;
associating a plurality of biomedical parameters of the user with execution of the plurality of activities to determine an effect of each activity on the plurality of biomedical parameters;
based on the association of the plurality of biomedical parameters with the execution of the plurality of activities and a predefined evaluation criteria, calculating a quality score for the user's schedule;
optimizing the user's schedule by performing a local search;
based on a generic ontology, automatically constructing an ontology comprising information associated with the plurality of activities and the plurality of biomedical parameters of the user; and
based on the constructed ontology, processing the natural language representation of the user's schedule.

2. The method of claim 1, wherein performing the local search comprises:
generating a set of candidate schedules within a neighborhood of predefined constraints and permitted modifications; and
selecting at least one of the candidate schedules based on the quality score.

3. The method of claim 1, wherein the plurality of activities and the plurality of biomedical parameters comprise activities and biomedical parameters related to one or more chronic medical conditions selected from the group consisting of: cardiovascular disease, dementia, kidney disease, and diabetes.

4. The method of claim 1, wherein the plurality of biomedical parameters are selected from the group consisting of: heart rate, blood pressure, blood sugar level, number of calories consumed, number of carbohydrates consumed, number of calories burned, and type and quantity of medication taken by the user.

5. The method of claim 1, wherein the generic ontology is constructed based, at least in part, on one or more lexical semantic databases.

6. The method of claim 1, wherein processing the natural language representation of the user's schedule is based on at least one of word sense disambiguation, named entity detection, named entity resolution, named entity inference, and word sense inference.

7. The method of claim 1, wherein the generic ontology comprises information associated with human activities and human biomedical parameters.

8. The method of claim 1, further comprising:
prompting the user to report on a progress of at least one activity of the plurality of activities.

9. The method of claim 1, further comprising:
allowing the user to modify one or more of the plurality of activities.

10. The method of claim 1, further comprising:
receiving, in real time, an input corresponding to one or more biomedical parameters of the user.

11. A computer system for natural language instantiation of a planning problem, the computer system comprising: one or more processors, one or more computer-readable memories, one or more computer-readable tangible storage devices, and program instructions stored on at least one of the one or more storage devices for execution by at least one of the one or more processors via at least one of the one or more memories, wherein the computer system is capable of performing a method comprising:
receiving, by a computer, a natural language representation of a user's schedule, the user's schedule comprising a plurality of activities;
associating a plurality of biomedical parameters of the user with execution of the plurality of activities to determine an effect of each activity on the plurality of biomedical parameters;
based on the association of the plurality of biomedical parameters with the execution of the plurality of activities and a predefined evaluation criteria, calculating a quality score for the user's schedule;
optimizing the user's schedule by performing a local search;
based on a generic ontology, automatically constructing an ontology comprising information associated with the plurality of activities and the plurality of biomedical parameters of the user; and
based on the constructed ontology, processing the natural language representation of the user's schedule.

12. The computer system of claim 11, wherein performing the local search comprises:
generating a set of candidate schedules within a neighborhood of predefined constraints and permitted modifications; and
selecting at least one of the candidate schedules based on the quality score.

13. The computer system of claim 11, wherein the plurality of activities and the plurality of biomedical parameters comprise activities and biomedical parameters related to one or more chronic medical conditions selected from the group consisting of: cardiovascular disease, dementia, kidney disease, and diabetes.

14. The computer system of claim 11, wherein the plurality of biomedical parameters are selected from the group consisting of: heart rate, blood pressure, blood sugar level, number of calories consumed, number of carbohydrates consumed, number of calories burned, and type and quantity of medication taken by the user.

15. The computer system of claim 11, wherein the generic ontology is constructed based, at least in part, on one or more lexical semantic databases.

16. The computer system of claim 11, wherein processing the natural language representation of the user's schedule is based on at least one of word sense disambiguation, named entity detection, named entity resolution, named entity inference, and word sense inference.

17. The computer system of claim 11, wherein the generic ontology comprises information associated with human activities and human biomedical parameters.

18. The computer system of claim 11, further comprising:
prompting the user to report on a progress of at least one activity of the plurality of activities.

19. The computer system of claim 11, further comprising:
allowing the user to modify one or more of the plurality of activities.

20. The computer system of claim 11, further comprising:
receiving, in real time, an input corresponding to one or more biomedical parameters of the user.
